(19) United States
(12) Patent Application Publication
(10) Pub. No.: US 2015/0351791 A1
(43) Pub. Date: Dec. 10, 2015

(54) SURGICAL INSTRUMENT

(71) Applicant: Ethicon Endo-Surgery, LLC, Guaynabo, PR (US)

(72) Inventors: James R. Morris, Sedalia, CO (US); James M. Morris, Sedalia, CO (US); Craig C. Morris, Sedalia, CO (US)

(21) Appl. No.: 14/830,320

(22) Filed: Aug. 19, 2015

Related U.S. Application Data
(63) Continuation of application No. 13/311,984, filed on Dec. 6, 2011, now abandoned.

(51) Int. Cl. A61B 17/32

(52) U.S. Cl. CPC A61B 17/320068; A61B 2017/00849

(57) ABSTRACT

An ultrasonically actuatable surgical blade comprises a metal blade and an elastomeric, biocompatible polymeric coating integral with the blade. The metal blade is usually titanium or a titanium alloy. The polymeric coating comprises a fluoropolymer resin and exhibits a Shore Hardness of 50 D to 60 D, an elongation at break of at least about 250 percent at a temperature in the range of about 20 EC. to about 200 EC. Optionally aluminum oxide powder can be dispersed in the coating.

### REFERENCE TO RELATED APPLICATIONS

The present application claims the priority benefit of U.S. provisional patent application serial no. 61/421,767, filed on Dec. 10, 2010.

### FIELD OF INVENTION

This invention relates to ultrasonic surgical instruments.

### BACKGROUND OF THE INVENTION

Ultrasonic instruments such as scalpels and the like are utilized to cut and coagulate tissue. In the case of a surgical scalpel that is provided with an ultrasonically actuatable blade, the blade is usually made of titanium and is vibrated at a frequency in the range of about 55,000 Hertz (Hz) to about 56,000 Hz and a displacement of about 70 to 80 microns. The blade operating temperature can be in the range of about 10 EC. to about 425 EC. In use, tissue tends to stick to the blade. Charring of the tissue, especially at the relatively higher operating temperatures, is encountered as well.

It would be desirable to minimize tissue sticking and charring as an ultrasonic surgical instrument is being used. The present invention satisfies these desires.

### SUMMARY OF THE INVENTION

An ultrasonically actuatable blade that substantially minimizes sticking to tissue and reduces eschar formation at the side of the incision is provided.

These features are achieved by an elastomeric, biocompatible coating integral with the working surfaces of the ultrasonically actuatable blade. The coating withstands transit and temperatures as low as −22 EC. and as high as 60 EC., and operating temperatures as high as 450 EC. Coated blades embodying the present invention also withstand ethylene oxide sterilization as well as e-beam and gamma sterilization.

A surgical scalpel embodying the present invention comprises an ultrasonically actuatable blade having a metal substrate bearing the aforementioned coating integral with the substrate and thus the blade. The coating is about 0.0005 to about 0.0025 inches thick, has a Shore Hardness value in the range of about 50 D to about 60 D, an elongation at break of at least about 250 percent at a temperature in the range of about 20 EC to about 200 EC, and is constituted by a fluoropolymer resin, preferably a resin which is a fused amalgam of fluorinated ethylene propylene, melamine resin, and a polyamide imide. The coating can further include aluminum oxide powder dispersed in the coating.

The coating can be applied to the ultrasonically actuatable blade by spray coating a blade having a surface that has a root mean square (RMS) surface roughness value in the range of about 15 to about 25 micro inches. For spray coating, the polymeric constituents of the aforementioned coating are dissolved in a non-aqueous solvent to provide a sprayable composition having a viscosity in the range of about 1000 centipoises to about 1500 centipoises, with or without having aluminum oxide powder suspended therein. If aluminum oxide is not present, the viscosity of the sprayable composition preferably is about 1000 to about 1200 centipoises. If the aluminum oxide is present in the sprayable composition, the viscosity of the sprayable composition preferably is about 1200 to about 1400 centipoises. A non-aqueous solvent such as isopropyl alcohol, and the like, can be used to adjust viscosity. The sprayable composition is deposited onto the substrate to a thickness of about twice the desired thickness for the final coating, dried at ambient temperature, and then at a temperature of at least about 150 EC. for about 20 minutes. After drying, the dried coating is heated at 330 EC. to about 360 EC., preferably at about 345 EC. for about 10 to about 45 minutes, preferably about 15 minutes to form an amalgam.

### BRIEF DESCRIPTION OF THE DRAWINGS

The novel features of the invention are set forth with particularity in the appended claims. The invention itself, however, both as to organization and methods of operation, may best be understood by reference to the following description, taken in conjunction with the accompanying drawings in which:

FIG. 1 is a fragmentary perspective view of a surgical scalpel provided with an ultrasonically actuatable blade coated with an elastomeric, biocompatible coating that embodies the present invention.

### DETAILED DESCRIPTION OF THE PREFERRED EMBODIMENTS

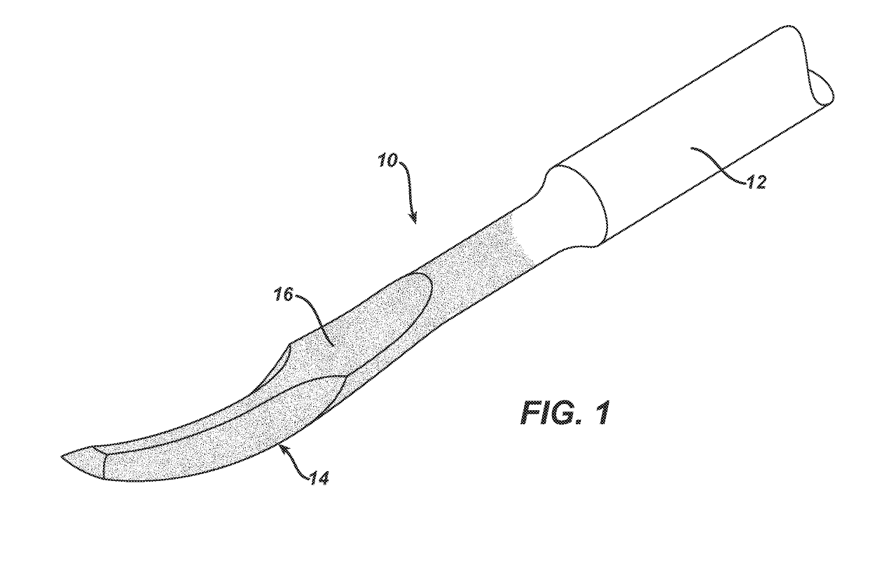

Referring to FIG. 1, surgical scalpel 10 is provided with ultrasonic transmission waveguide 12 to which is coupled an ultrasonically actuatable blade or ultrasonic end effector 14. The elastomeric, biocompatible coating 16 covers the blade 14 and is integral with blade 14 which is usually made of titanium or a titanium alloy. The coated blade has a Shore Hardness (ASTM D2240) value in the range of about 50 D to about 60 D.

Coating 16 overlies a surface of blade 14 that exhibits a root mean square (RMS) surface roughness value in the range of about 15 to about 25 micro inches, preferably about 20 micro inches, which is equivalent to an arithmetic average surface roughness of about 16 to about 18 micro inches. If necessary, the surface of blade 14 can be roughened prior to spray coating by micro-abrasive blasting using compressed air and an abrasive powder such as aluminum oxide, sodium bicarbonate, silicon carbide, crushed glass, and the like, or in any other convenient manner that imparts the desired roughness characteristics to the blade surface prior to spray coating.

When an abrasive powder is utilized to roughen the blade surface, the abrasive powder can have a particle size preferably in the range of about 30 to about 75 microns, more preferably about 50 microns.

After micro-abrasive blasting the loose material on the so treated surface can be removed prior to spray coating by a high pressure water spray, or in any other convenient manner.

The solvent for the sprayable coating composition includes a non-aqueous solvent such as naphta, methylisobutyl alcohol, n-butyl alcohol, methyl pyrrolidone, and mixtures thereof. The non-aqueous solvent is selected having a relatively high vapor pressure at ambient temperature so that the solvent can be readily removed from the coated blade by drying at ambient temperatures.

An important physical property of the present coatings is elongation at break. The present coatings exhibit at least a 250 percent elongation at break over a temperature range of about 20 EC. to about 200 EC., preferably an elongation of about 280 percent to about 350 percent at the aforesaid temperature range. This elongation permits the concurrent flexing of the adhered coating together with the blade when subjected to the ultrasonic vibrations.

Typical physical characteristics of a coating that embodies the present invention are set forth in Table I, below:

TABLE I
Physical Characteristics of FEP Resin Coating

| | | Rating/ | ASTM |
|---|---|---|---|
| Measure | Unit | Value | Standard |
| Nonstick | — | E | None |
| Chemical Resistance | — | E | None |
| Abrasion Resistance | — | G | None |
| Salt Spray Resistance | — | E | None |
| Water Absorption | % | <0.01 | D570 |
| Coefficient of Friction - Kinetic | — | 0.08 | D1894 |
| Coefficient of Friction - Static | — | 0.2 | |
| Specific Gravity | — | 2.15 | |
| Melt Point | EF | 500 | |
| Hardness | Shore D | 55 | D2240 |
| Maximum Continuous | EC | 205 | None |
| Temperature | EF | 400 | |
| Thermal Conductivity | (Btu) (in)/(ft²) | 1.35 | |
| | (hr) (EF) | | |
| Dielectric Strength | V/mil | 2000 | D149 |
| (short-term 10-mil film) | | | |
| Surface Resistivity | ohm/square | 1.0E18 | D257 |
| Volume Resistivity | Ohm-cm | 1.0E16 | D257 |
| Tensile Strength | MPa at 23 EC | 23 | D1708 |
| Elongation at Break | % at 23 EC | 325 | D1708 |

The fluoropolymer resins suitable for use in practicing the present invention include the polytetrafluoroethylene resins such as Teflon® PTFE and the like, the fluorinated ethylene propylene copolymer resins such as Teflon® FEP and the like, and the perfluoroalkoxy resins such as Teflon® PFA and the like. Particularly preferred for the present purposes is Teflon® S fluoropolymer resin No. 959-203 which comprises fluorinated ethylene propylene resin, melamine resin, and a polyamide imide polymer. This particular resin is commercially available from E. I. DuPont de Nemours Co., Fluoroproducts, Wilmington, Del. 19890 as a solution in a non-aqueous solvent mixture comprising methyl isobutyl ketone, formaldehyde, n-butyl alcohol, methyl pyrrolidone and VM&P Naphtha. The fluorinated ethylene propylene (FEP) and the polyamide imide are present in the fluoropolymer preferably in a respective volume ratio of about 2:3. Upon heating, the polymeric constituents form an amalgam.

Aluminum oxide powder in the elastomeric coating is optional. The aluminum oxide powder in the fluoropolymer resin is desirable when the coating thickness is greater than about 0.0008 inch. The aluminum oxide powder can be dispersed substantially uniformly throughout the coating, or the concentration of the aluminum oxide power in the coating can vary along its thickness, with the relatively higher powder concentration being closer to the surface of the blade.

The aluminum oxide particles present in the elastomeric coating also provide anchor points that serve to increase adherence of a top coating layer that contains little or no aluminum oxide powder.

The elastomeric coating embodying the present invention can be constituted by more than one layer, with the coating layer contiguous with the surface of the titanium blade having a relatively higher concentration of aluminum oxide than an intermediate or top layer of the coating. In this manner, a concentration gradient of aluminum oxide powder can be provided in the elastomeric coating, if desired, by multiple spraying and drying cycles prior to final amalgam formation.

One preferred embodiment comprises an ultrasonically actuatable blade provided with an elastomeric coating that has a base layer containing aluminum oxide power and a top layer over the base layer that contains no aluminum oxide powder.

The relative amounts of FEP and polyamide imide can be varied to adjust the properties of the coating. An increase in the relative amount of FEP provides enhanced lubricity whereas a decrease in the amount of FEP enhances adhesion of the coating to the blade, if desired.

The aluminum oxide powder can have a mean particle size in the range of about 0.5 microns to about 5 microns, preferably about 1 micron.

The amount of aluminum oxide powder present in the coating composition preferably is in the range of about 0.1 to about 5 percent by weight of the sprayable composition solids.

The sprayable composition, which includes the aluminum powder substantially uniformly dispersed therein has a viscosity in the range of about 1200 centipoises to about 1400 centipoises. During the spray coating process the blade is coated with a layer having a thickness of about twice the desired final coating thickness. This layer is then air-dried at ambient temperature for about 15 minutes to remove some of the solvent and then at a temperature of at least 150 EC. for at least about 20 minutes to remove the rest of the solvent. These process steps can be repeated, if desired, to provide a relatively thicker coating or to adjust the distribution of aluminum oxide powder in the final coating. To form the amalgam, the dried coating is heated at about 330 EC. to about 360 EC., preferably about 345 EC. for a time period of about 10 to about 45 minutes, preferably about 15 minutes. A heating temperature below about 330 EC. is too low for amalgamation. A heating temperature above about 360 EC. results in an undesirably brittle coating.

If desired, when particulate materials can be introduced into the coating to achieve greater wear resistance, modulate heat transfer, modulate conductivity, and the like. For example, yttrium powder can be added to the sprayable composition for greater wear resistance of the final coating as well as enhanced thermal insulation. Similarly, tungsten powder can be added to the sprayable composition for enhanced heat transfer.

The present invention is illustrated by the following Example.

### Example 1

### Manufacture of a Coated, Ultrasonically Actuatable Blade

A conventional ultrasonically actuatable blade made of titanium is micro-blasted with aluminum oxide having a mean particle size of about 10 microns at an air pressure of about 70 to 80 psig to obtain a RMS surface roughness of about 20 micro inches. The blade is then rinsed with a high pressure water spray at a water pressure of about 250 psig.

A sprayable coating composition is prepared by adding aluminum oxide powder (1 micron mean particle size; about 3 percent by weight) to a fluoropolymer resin (DuPont No. 906-203) with stirring to produce a composition having the aluminum oxide powder substantially uniformly dispersed therein and a viscosity of about 1400 centipoises. This sprayable composition is then applied to the blade with an automatic spray gun (Spraying Systems Type 1/8VAU-SS and B1/8VAU-SS Variable Spray Autojet Automatic Air Atomizing needle, size 0.0340″). Approximate nozzle size is 0.0342″ and approximate air cap size 0.125″. The coating composition is atomized at 30 psig and applied at about 2.7 psig. The fan pattern is adjusted to about 32 psig.

During spray coating, the blade is rotated within the spray pattern at a rate of about 395 revolutions per minute (RPM).

The spray application is continued until a layer about 0.005″ thick is deposited on the blade. The blade is then air dried for about 15 minutes, and then heated at about 150 EC. for about 20 minutes to remove the rest of the solvent. The dried coating is thereafter heated at 345 EC. for about 15 minutes; and cooled to ambient temperature.

The coated blade produced in the foregoing manner has a coating thickness of about 0.00125″ on each side of the blade. The coating has a Shore Hardness of 55 D. In use, the coated blades exhibit significantly less tissue sticking and eschar buildup.

The foregoing discussion and the Example are illustrative, and are not to be taken as limiting. Still other variants within the spirit and scope of this invention are possible and will readily present themselves to those skilled in the art.

I claim:

1. A surgical scalpel which comprises an ultrasonically actuatable blade and an elastomeric, biocompatible polymeric coating integral with said blade and defining a blade-coating interface;
the coating having a thickness in the range of about 0.0005 inch to about 0.0025 inch, a Shore Hardness value in the range of about 50 D to about 60 D, an elongation at break of at least about 250 percent at a temperature in the range of about 20 EC. to about 200 EC., and comprising a fluoropolymer resin.

2. The surgical scalpel in accordance with claim 1 wherein the coating includes aluminum oxide power dispersed therein.

3. The surgical scalpel in accordance with claim 1 wherein the aluminum oxide powder is present in an amount in the range of about 0.1 percent to about 5 percent by weight, based on the weight of the coating.

4. The surgical scalpel in accordance with claim 1 wherein the aluminum oxide powder has a mean particle size in the range of about 0.5 to about 5 microns.

5. The surgical scalpel in accordance with claim 1 wherein the aluminum oxide powder has a mean particle size of about 1 micron.

6. The surgical scalpel in accordance with claim 1 wherein the blade comprises titanium with root mean square (RMS) surface roughness value in the range of about 15 to about 25 micro inches at the blade-coating interface.

7. The surgical scalpel in accordance with claim 1 wherein the blade comprises titanium with a root mean square surface roughness value of about 20 micro inches at the blade-coating interface.

8. The surgical scalpel in accordance with claim 1 wherein the blade comprises titanium and the arithmetic average of the surface roughness of the blade surface is in the range of about 16 to about 18 micro inches.

9. The surgical scalpel in accordance with claim 1 wherein the fluoropolymer resin comprises fluorinated ethylene-propylene, melamine resin, and polyamide imide, and the fluorinated ethylene-propylene and the polyamide imide are present in the coating in a respective volume ratio of about 2:3.

10. The surgical scalpel in accordance with claim 1 wherein the blade has a Shore Hardness value of about 55 D.

11. The surgical scalpel in accordance with claim 2 wherein the aluminum oxide powder is distributed substantially uniformly in the coating.

12. The surgical scalpel in accordance with claim 2 wherein concentration of the aluminum oxide powder in the coating is relatively higher in vicinity of the blade-coating interface.

13. The surgical scalpel in accordance with claim 1 wherein the fluoropolymer resin is a fused amalgam of fluorinated ethylene propylene, melamine resin and a polyamide imide.

14. The surgical scalpel in accordance with claim 1 wherein the fluoropolymer resin is a fused amalgam of fluorinated ethylene propylene, melamine resin, and a polyamide imide and contains aluminum oxide powder.

15. The surgical scalpel in accordance with claim 14 wherein the aluminum oxide power is distributed substantially uniformly within the amalgam.

16. The surgical scalpel in accordance with claim 14 wherein the aluminum oxide powder is present at a relatively higher concentration at the blade-coating interface.

17. A method of applying an elastomeric, biocompatible polymeric coating to an ultrasonically actuatable surgical blade which comprises the steps of:
combining a solution of a fluoropolymer in a non-aqueous solvent with aluminum oxide powder to obtain a substantially uniform dispersion of the aluminum oxide powder in the solution to provide a suspension having a viscosity in the range of about 1000 centipoises to about 1400 centipoises;
spray coating the blade, having a root mean square (RMS) surface roughness value in the range of about 15 to about 25 micro inches, with the suspension to provide a substantially uniform deposit of at least about 0.005 inches thick on the blade;
removing the solvent from the deposit by evaporation at ambient temperature and then heating the deposit at a temperature of about 150 EC. for at least about 20 minutes to produce a dried deposit on the blade; and
thereafter heating the dried deposit at about 330 EC. to about 360 EC. for about 10 to about 45 minutes.

18. The method in accordance with claim 10 wherein the blade is micro-blasted with aluminum oxide powder having a mean particle size of about 50 microns at about 70 pounds per square inch gauge prior to the spray coating step.
